(19) United States
(12) Patent Application Publication
(10) Pub. No.: US 2025/0153001 A1
(43) Pub. Date: May 15, 2025

(54) ANKLE MOVEMENT TRACKING BAND AND ASSOCIATED METHODS

(71) Applicant: Marco GUIROLA, San Salvador (SV)

(72) Inventor: Marco GUIROLA, San Salvador (SV)

(21) Appl. No.: 18/941,304

(22) Filed: Nov. 8, 2024

Related U.S. Application Data
(60) Provisional application No. 63/548,014, filed on Nov. 10, 2023.

(51) Int. Cl. A63B 24/00

(52) U.S. Cl. CPC A63B 24/0062; A63B 2220/40; A63B 2225/02; A63B 2225/50

(57) ABSTRACT

An ankle movement tracking band includes an electronic device configured to be worn on an ankle region of a user. The electronic device has at least one motion sensor configured to capture sensor data of movement of the ankle region of the user. A processor is coupled to a memory for storing calibration data, and the processor is configured to receive and analyze the sensor data to determine whether the sensor data matches the calibration data indicating the user performed a soleus pushup. The motion sensor includes at least one of an accelerometer, a gyroscope, and a Global Positioning System (GPS) receiver. The electronic device also includes a wireless communication transceiver configured to connect to a device and transmit the sensor data to the device to display a count of the soleus pushups performed by the user.

### CROSS-REFERENCE TO RELATED APPLICATIONS

This application claims the benefit of U.S. provisional application No. 63/548,014 filed Nov. 10, 2023, which is hereby incorporated herein in its entirety by reference.

### FIELD

The present invention relates to exercise accessories, and, more particularly, to an ankle movement tracking band and associated methods.

### BACKGROUND

Contemporary lifestyles are often sedentary and involve significant periods of inactivity, including while sitting at a desk, laying on a bed or couch, or sitting in cars, trains and buses while commuting. Such sedentary lifestyles, which may be associated with office settings, school settings, the elderly, and other comparable environments, may pose health risks, including serious conditions. Such health risks associated with significant periods of inactivity may include loss of muscle strength, loss of mineral content in bones, changes to a person's metabolism, weight gain, poor blood circulation, hormonal imbalances and certain inflammatory illnesses and conditions. Yet, it is often challenging to maintain a minimum level of exercise that can effectively counter the foregoing health risks associated with sedentary environments and lifestyles.

Activation of the soleus muscle, a small but powerful postural muscle in the back part of the lower leg or calf, may provide significant health benefits. Exercising the soleus muscle can be a great exercise alternative as it may sustain metabolism and/or improve glucose regulation in humans regardless of age and/or gender. Such potential may be realized when the soleus muscle is activated by completing soleus pushups, i.e., contractions and expansions of the soleus muscle. Importantly, such exercises of the soleus muscle may be achieved while sitting and can be an ideal form of exercise for individuals that otherwise maintain a sedentary lifestyle. Adults that are able to achieve a minimum number of soleus pushups over a predetermined timeframe, for example, 1-3 hours, may achieve beneficial results compared to those individuals that sit inactive during a comparable timeframe. Such soleus muscle exercises or pushups are believed to increase whole-body carbohydrate oxidation when compared to periods of inactivity while sitting. In turn, this may at least partially improve a systemic metabolism. Even subtle increases in the energy expended during periods of activity of the soleus muscle are believed to help sustain muscle metabolism. It is further believed that a targeted activation of the soleus muscle could meaningfully impact whole-body metabolism over prolonged periods of activity. Thus, there are appreciable benefits that may be achieved by exercising the soleus muscle, or more simply by completing heel lift exercises.

Against this backdrop, there is a need in the industry to provide for a solution that can motivate individuals to perform soleus pushups or heel lifts while sitting. A benefit would be realized by providing a device that is capable of tracking the number of heel lifts that a user performs while sitting. Another advantage would be realized if such a device would be able to ascertain such exercise data in real time and would transmit it to a paired device(s) so that it could be displayed, including in a community setting. An even further advantage would be realized if the paired device could run a mobile application that could implement gamification and/or virtual reality module. That is, the paired device may show a progression through a geographical environment according to the level of exercise performed by the user. An even further advantage would be realized if the device could provide feedback, e.g., haptic feedback, a sound alarm, a visual alarm, to the user upon completion of certain milestones or upon periods of inactivity in order to promote engagement. Yet a further advantage would be realized by providing a device with a paired mobile device that could visually display the progress of a group of individuals, including in the same virtual reality environment.

### SUMMARY

The present invention is directed to an ankle movement tracking band. The tracking band can be worn on the ankle region of a user, and which may be used to monitor oscillatory heel-lift exercises otherwise also known as soleus pushups. The tracking band includes an electronic device, where the electronic device includes at least one motion sensor configured to capture sensor data of movement of the ankle region of the user. A processor is coupled to a memory for storing calibration data, and the processor is configured to receive and analyze the sensor data to determine whether the sensor data matches the calibration data indicating the user performed a soleus pushup. The calibration data correlates to vertical, oscillatory movements of performing the soleus pushup. The motion sensors include at least one of an accelerometer, a gyroscope, and a Global Positioning System (GPS) receiver. The electronic device also includes a wireless communication transceiver configured to connect to a device such as a mobile device, and transmit the sensor data to the device.

The device is configured to connect to a network to transmit the sensor data to a remote device for processing. The electronic device further includes an antenna coupled to the wireless communication transceiver. In addition, the tracking band includes a soleus pushup counter module that is configured to count a number of soleus pushups performed by the user, and an ankle mount, where the electronic device is secured to the ankle mount. The ankle mount includes a receiver for removably attaching a decorative component to cover the electronic device. The ankle mount may include a strap configured to encircle the ankle of the user or a clip configured to secure to clothing proximate to the ankle of the user.

The electronic device may also include a haptic device, where the haptic device is configured to vibrate in response to receiving an alert from the processor. In addition, the tracking band may include an ancillary second electronic device configured to be worn on the other ankle of the user, where the ancillary second electronic device has at least one respective motion sensor configured to capture respective sensor data of movement of the other ankle region of the user.

In another aspect, a method of tracking ankle movement of a user is disclosed. The method includes placing a tracking band on an ankle region of the user, detecting movement by an electronic device of the tracking band, and transmitting sensor data of the movement to a device. The method also includes analyzing the sensor data to determine if a soleus pushup was performed, increasing a count of a soleus pushup count when the sensor data matches with calibration data that the soleus pushup was performed, and disregarding the sensor data when the sensor data does not match with the calibration data the soleus pushup was not performed. The method may also include placing a second tracking band on an other ankle region of the user, and displaying the soleus pushup count on the device.

As the user completes oscillatory upward and downward soleus pushup cycles, the motion sensors are configured to ascertain and interpret data characteristics associated with each cycle. These data characteristics may include the real-time position of the ankle, the timeframes involved, and calories expended in each oscillatory cycle, for example. This ascertained sensor data is stored on the electronic device or on the paired mobile or stationary device. The ascertained sensor data may be wirelessly transmitted via Bluetooth, WiFi, etc. to the paired mobile or stationary device(s).

The device may be configured to implement a downloadable application to enable or execute various of the operative features of the present invention. As such, the device may receive, processes, interpret, store or display the ascertained soleus pushup data. Displayed soleus pushup data may include real-time or historical pushup totals. The tracking band through the application, may show real-time data characteristics associated with a current or past soleus pushup session. Such data may include the total number of soleus pushup cycles completed, equivalent calories spent during such cycles or advancement on a geographical route or visual environment.

Additional features of the present invention comprise gamification features that are intended to motivate the user(s) and enhance engagement while exercising the soleus muscle. That is, it is within the scope of the present invention to provide a virtual reality module that can be downloaded and/or uploaded to, and viewed on, the device or an ancillary monitor. In the virtual reality module, it is contemplated that a user(s) may view a visual representation of the user's movement along a visual environment, e.g., a hike, according to the number of soleus pushup cycles that the user completes. Said differently, as the user completes cycles of soleus pushups, a visual representation of a physical progression through a specific environment may be shown on the mobile device screen or ancillary device or monitor. Such visual representation of a progression is intended to be not only rewarding to the individual user, but may also be shared with other users. The present invention contemplates that social motivation may be enhanced in a community setting or otherwise by participating in group challenges and monitoring collective progress.

The present invention also contemplates providing rewards, including in the form of new environments, modules or avatar accessories after reaching certain milestones. Additionally, and as briefly mentioned above, the present invention contemplates community settings where groups can collectively progress through environments based on their completed pushup cycles and can view data associated with their own progression and the progression of other users. Users can also upload or acquire additional environments and content either through a real money purchase or a reward exchange.

Additional features of the present invention comprise providing a tracking device configured to provide feedback to the user(s). For example, alerts via haptic vibration, audio tones, or visual indicators, e.g., lights, may be triggered based on completion of certain milestones, based on a performance that falls below certain minimum thresholds or based on other conditions. Such alerts may be configured to provide feedback to the user in the form of lights, sounds and haptic vibrations either on the electronic assembly itself or via the paired mobile device. Such alerts may also be configured to allow the user to achieve an exercise balance between the left and right ankle regions.

### BRIEF DESCRIPTION OF THE DRAWINGS

The aspects and the attendant advantages of the embodiments described herein will become more readily apparent by reference to the following detailed description when taken in conjunction with the accompanying drawings wherein:

FIG. 1 is a side perspective view of an ankle movement tracking band placed on an ankle region of a user and in which various aspects of the disclosure may be implemented;

FIG. 2 is a side perspective view of the ankle movement tracking band of FIG. 1 paired to a mobile device;

FIG. 3 is a side perspective view of an ancillary second ankle movement tracking band placed on the other ankle region of a user;

FIG. 4 is a side perspective view of an ankle mount of the ankle movement tracking band placed on the user;

FIG. 5 is a side perspective view of the ankle movement tracking band of FIG. 4 paired to the mobile device;

FIG. 6 is a cross-sectional side view of the ankle movement tracking device of FIG. 1;

FIG. 7 is a detailed view of an ankle mount of the ankle movement tracking band;

FIG. 8 is a perspective view of the ankle mount of FIG. 4; and

FIG. 9 is a flow chart of a method of tracking ankle movement of a user.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

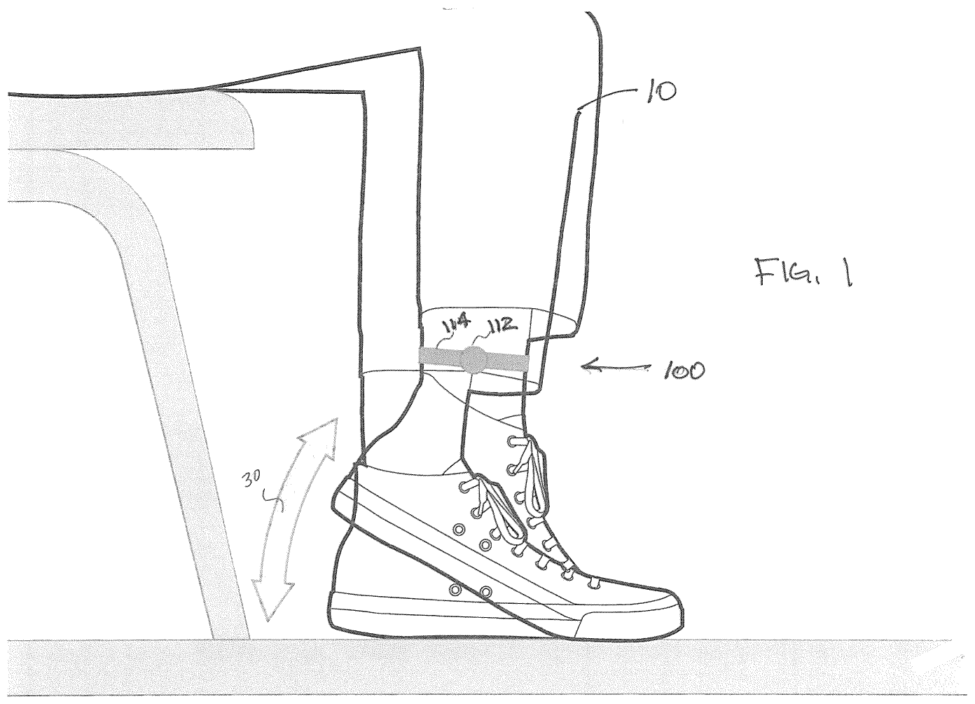

With initial reference to FIGS. 1-5, the present invention is directed to an ankle movement tracking band 100, which is configured to track movement of the ankle or lower part of the leg of a user while the user is in a sitting position. The ankle movement tracking band (also referred to herein as “tracking band”) 100 includes an electronic device 112 having at least one sensor and a wireless communication transceiver 126. The electronic device 112 is configured to communicate with a mobile device 20, or stationary device, to ascertain, track or otherwise monitor, and interpret a movement of the ankle or an ankle region of the leg of the user while sitting. The ankle region of the user is an area of the leg from the ankle to below the knee. The movement of the knee is not consistent with counting a soleus pushup. Accordingly, the accuracy of counting soleus pushups is improved by placing the tracking band 100 proximate the ankle rather than the knee of the user. It is within the scope of the present invention that the user wear the tracking band 100 while sitting and that the electronic device 112 ascertain at least an upward and downward movement thereof during a soleus pushup cycle, which will be explained in more detail below.

The electronic device 100 may be disposed onto or attached to an ankle(s) or lower part of the leg of a user, or may be attached to a wearable item around the ankle area, e.g., the electronic device 100 may be attached to a shoe, pant sleeve, sock, etc. As will also be explained hereinafter, one electronic device 100 may be attached to one ankle or leg, whereas an ancillary or second electronic device 100 may be attached to the other ankle or leg.

As shown throughout FIGS. 1-3, the tracking band 100 includes the electronic device 112 disposed on an ankle mount 114. The ankle mount 114 may comprise an adjustable band, an elastic band, a belt, strap or other comparable structure that may be used to secure the electronic device 100 around the ankle of the user 10. The electronic device 112 is generally disposed on the ankle mount 114, including on an exterior side thereof, via one of various possible attachment mechanisms. Such attachment mechanism may include, without limitation, adhesives, clips, snap-on mechanisms, magnets, mounts, brackets, connectors, configured for insertion of the electronic device 112, sewing arrangements and other comparable attachment mechanisms. The electronic device 112 may also be inserted into a configured pocket in a sock, shoe or other garment of the user.

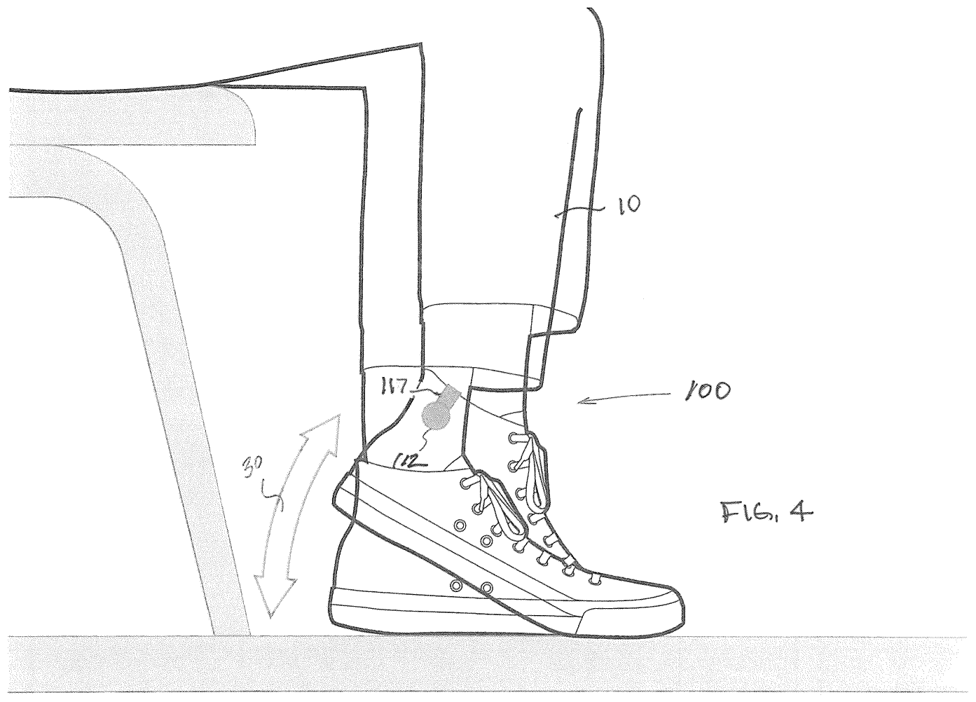

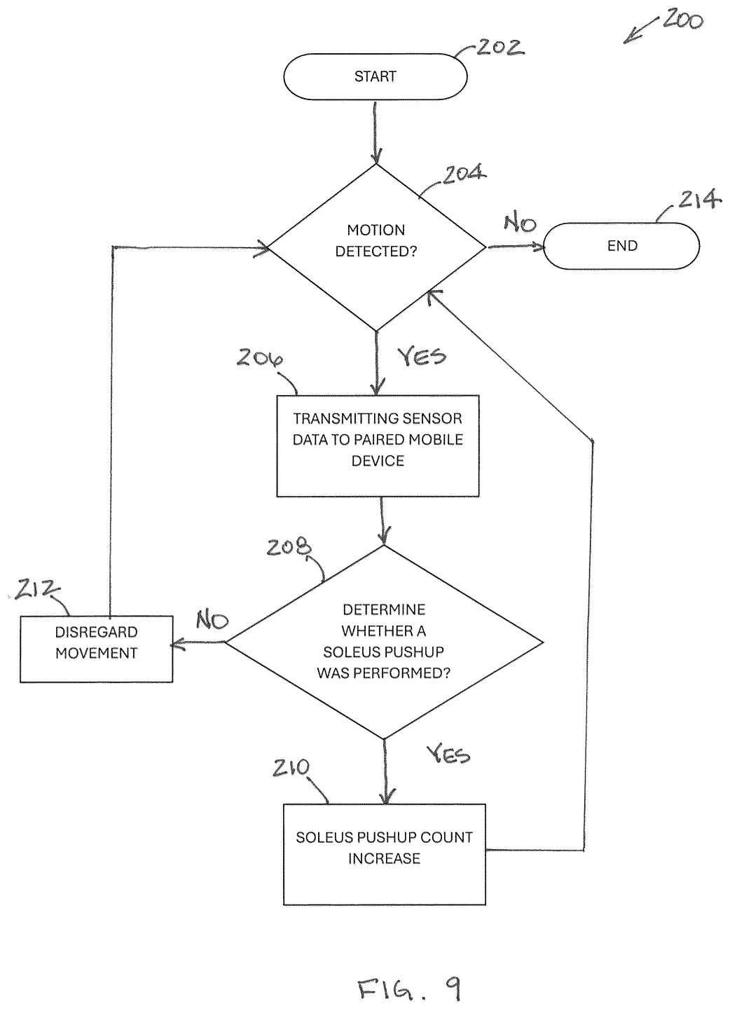

For example, in the illustrative embodiments of FIGS. 1-3, the ankle mount 114 may comprise a band that may be disposed around the ankle or lower leg part of the user 10. In another aspect shown in FIGS. 4, 5, and 8, the tracking band 100 may comprise a clip mount 117. Generally, the clip mount 117 will be attached to a wearable item, e.g., shoe, sandal, pant sleeve, etc., around the ankle area. A clip mount(s) 117 may be advantageous for situations where the user 10 prefers such versatility or for situations where it is not desirable or practical to attach an ankle mount 114 directly to the ankle(s) or lower part of the leg(s) of the user 10.

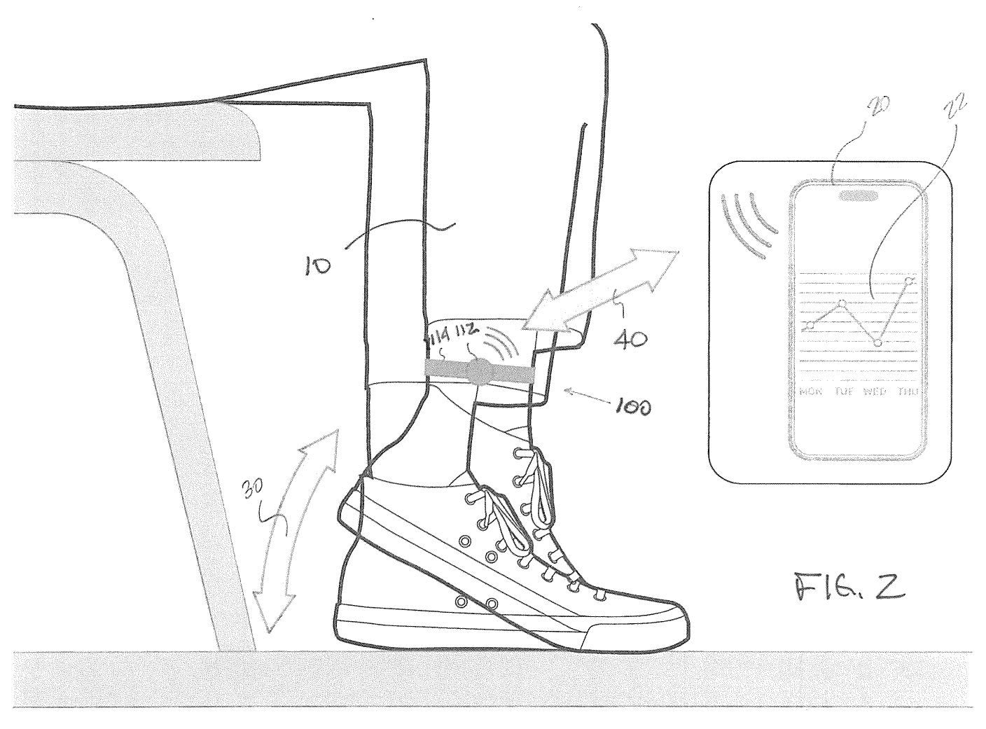

As is shown in FIGS. 2 and 5, and as briefly mentioned above, the tracking band 100 includes the electronic device 112 operatively configured with a mobile device 20 to track and interpret data associated with at least the vertical, oscillatory movement of the ankle or leg of the user 10. The electronic device 112 itself may comprise circuitry configured to ascertain the number of partial and/or complete soleus pushups that the user 10 completes.

As used herein, the term “soleus pushup” refers to an oscillatory movement of the ankle or lower part of the leg of the user 10, upward and downward, which is at least partially caused, respectively by a contraction and expansion of the soleus muscle. The soleus pushup movement is indicated by the arrow 30. Said differently, a “soleus pushup” involves a minimum upward and corresponding downward motion of the ankle or lower part of the leg of the user 10. Further, as used herein a “soleus pushup cycle” refers to a partial or full contraction and a partial or full expansion movement of the soleus muscle, or what may also be referred to as a “heel lift” maneuver.

The electronic device 112 generally comprises a sensor configured to ascertain the position and movement thereof at least in the vertical direction. Such detection of the movement by the sensor, which is described in more detail below, reciprocates the movement of the corresponding ankle, foot or lower part of the leg(s) of the user 10 where the electronic device 112 is attached. As such, the tracked or ascertained position(s) or movement of the electronic device 112 should be representative of the reciprocal, corresponding position(s) or movement of the ankle or lower part of the leg of the user 10. As the sensor ascertains and gathers data associated with each soleus pushup cycle(s), such data may be stored, temporarily or permanently, e.g., on a memory unit disposed within the electronic device 112. Additionally, and with reference to FIGS. 2 and 5, the ascertained data associated with soleus pushup cycle is wireless transmitted 40 to a paired device 20.

With reference again to FIGS. 2 and 5, the tracking band 100, including the sensor 112 may be operatively configured or paired with the device 20 via a variety of wireless capabilities. Such pairing is intended at least to transmit sensor data associated with completed soleus pushup cycles. Such wireless capabilities may include, without limitation, near-field communication (NFC), a WiFi connection, a Bluetooth® connection, radio frequency identification (RFID), cellular (3G, 4G, 5G), Wireless Personal Area Networks (WPAN), Wireless Local Area Networks (WLAN), Wireless Wide Area Networks (WWAN), Wireless Metropolitan Area Networks (WMAN), etc.

In turn, the device 20 may comprise a processor or central processing unit (CPU) operatively configured with the electronic device 112 to receive, interpret and or process the data associated with each soleus pushup cycle. Optionally, such a processor may be built into the electronic device 112, and may be operatively configured with the sensor in a similar fashion.

Additionally, the device 20 may be operatively connected with a data network, or with other devices to transmit data characteristics associated with one or more completed soleus pushup cycle between different devices 20. Such operative communication between the device 20 and the data network may be enabled via local area network (LAN), wired area network (WAN), ZIGBEE, BACNET, RS-485, Serial, Z-WAVE, MODBUS, the Internet, or combinations thereof. The device 20 itself may comprise various types of mobile devices such as mobile telephones, tablets or smart watches, e.g., Android or Windows-enabled devices, iPhone or iOS enabled devices, etc. As used herein, term “device” is interchangeable with the term “mobile device.” Furthermore, the device 20 may also comprise memory capabilities, including volatile or non-volatile storage, random access memory (RAN), read-only memory (ROM), etc. Additionally, various of the operative features described herein may be implemented by the device by way of a computer program or other interpretable language, including but not limited to, C, C++, HTML, Java, Dart, MATLAB, Swift, Rust, PHP, XHTML, etc.

With even further reference to FIGS. 2 and 5, the present invention contemplates that the device 20 comprises a screen 22 configured to display information relating to the ascertained and/or interpreted data characteristics associated with a soleus pushup session. For example, a user 10 may download a mobile application to the device 20 to access various of the operative features of the present invention.

Alternatively, such features may be accessed through a web browser. Such a downloadable mobile application or web browser may be displayed on the screen 22 of the device 20, and may be enabled, for example, via an application programming interface (API) or a graphical user interface (GUI). The downloadable mobile application or interface may be accessed to display data associated with a “soleus pushup session,” e.g., a predetermined number of fully or partially completed soleus pushup cycles, or any given number of soleus pushup cycles executed in a predetermined timeframe. As will be explained hereinafter, the mobile application may also be used to display a virtual environment and/or other gamification features of the present invention. Data associated with a soleus pushup session and/or a soleus pushup cycle(s) may be transmitted from the electronic device 112 to the device 20 on a “real-time” basis, such that the user 10 may see the number of soleus pushups or time associated with any particular session. Further, data associated with a soleus pushup session may also be represented as total calories expended during a session, rates of movement or oscillation, equivalent distance traveled during a session, or another comparable measure of work performed by the user 10 during a soleus pushup session.

Additional features of the present invention comprise a virtual reality and/or gamification features that may be implemented on the device 20, including via the downloadable mobile application or web browser. For example, a user 10 may be able to download a virtual reality module comprising a particular virtual and/or interactive theme or visual environment. Examples of such modules may consist of a visual representation, including in 3D and/or virtual reality (VR), of a visual progression through a geographical region, e.g., a hike through the Sierra Nevada, the Appalachian Trail or another geographic location. Such a theme or environment may include a visual representation of a progression, including in real-time, through the particular environment according to the number of soleus pushup cycles that the user 10 completes.

For example, as the user 10 completes any given number of soleus pushup cycles, such progress may be represented as a virtual user movement along the particular or predetermined environment, e.g., physically advancing through a trail or climbing a mountain. Said differently, it is contemplated that the user 10 be able to advance through a particular environment as the user 10 completes soleus pushup cycles. For example, as the tracking band 100 ascertains data characteristics associated with a number of completed soleus pushup cycles, data characteristics may be interpreted and correlated to an expended calorie or glycogen count, for example. In turn, such calorie or glycogen count may correlate to a predetermined progress along the particular environment, e.g., visual progress along Appalachian Trail.

Such progress along a particular environment may be tied to various prizes or rewards, including additional content for download, additional challenges, monetary rewards, etc. It is further contemplated that such progress through a particular environment or module may be displayed on the screen 22 of the device 20, e.g., via the mobile application or web browser, or may be projected onto another screen or monitor.

Further features of the present invention contemplate allowing users to download and/or upload additional content via the device 20. For example, the present invention contemplates allowing a user 10 to purchase additional content, e.g., additional VR modules or environments. Further, the user 10 may download such additional content to the device 20, including via the mobile application or web browser. Accordingly, the mobile application may be operatively configured with payment tools or platforms that may enable the user 10 to purchase and/or download additional content via the mobile device 20. As a further example, the user 10 may also exchange available rewards for additional content, or may exchange available rewards for reduced pricing on additional content.

As an even further example, the user 10 may have the option to upload content, e.g., additional environments that the user 10 or other users may be able to purchase, exchange, download and/or access on their own devices 20. As such, various users may individually or simultaneously progress through one or more of multiple available environments, including in a community setting. Such a community setting may involve a family group, a friend group, a school group, random groups, elderly groups, office groups, assisted facility groups, special interest groups and/or combinations thereof. That is, multiple users can visually see their own progress through a particular environment and can also see and track the progress of other individuals through the same environment.

Furthermore, the present invention contemplates that data associated with progress of each individual along an environment, or the progress of the group, may be communicated in real-time to other users. Such real-time communication may be visually represented on the downloadable mobile app, but also through other platforms, e.g., text messaging, alarms reproduced on the electronic device 112, etc.

Accordingly, such gamification features of the present invention, including the community setting, rewards, and additional content, are intended to serve as motivation to enhance participation and collaboration between various users. For example, some environments may allow personal or individual metrics, e.g., regarding progress through an environment, that may impact the overall metrics of a specific community or group participating in the same environment. As a further example, visual progress or completion of a particular environment may be a function of the overall progress, or collective number of soleus pushup cycles, of the group and not merely each individual.

Additional features of the present invention may include configuring the electronic device 112 to generate alerts in order to enhance the user experience and convey certain information to the user 10. The electronic device 112 may be provided with various components that can provide feedback to the user 10, e.g., haptic feedback by way of vibration or force feedback. Such feedback may be selectively configured to alert the user 10 of the occurrence of a predetermined condition.

Such a predetermined condition may involve, for example, a predetermined period of inactivity, a predetermined number of completed soleus pushup cycles that falls below a given threshold, including within a given timeframe either for the individual user 10 or collectively for a group. Conversely, such a predetermined condition may also involve meeting certain predetermined goals or targets of soleus pushup cycles or metrics during a given timeframe or after partial or full completion of a virtual module or environment or achieving a milestone.

As a further example, such alerts and feedback may be selectively implemented to alert the user 10 to complete more soleus pushup cycles on one leg versus the other to achieve an exercise balance between them. Additionally, the screen 22 of the device 20 may be used to display visual representation of characters, videos, events, images or other visual or sound representations thereof upon the occurrence of such predetermined conditions. For example, the device 20 may be configured to receive video and other images that can be used to stimulate the soleus pushup experience. For example, the user completing a soleus pushup cycle may correlate with moving through the video on the screen 22. Content creators are also incentivized to submit content that can be used with the device 20 and the tracking band 100.

In lieu of such haptic features, the present invention also contemplates that the electronic device 112 and/or the device 20 may be provided with hardware and/or software to provide feedback to the user 10 by way of a sound alarm, e.g., speaker feedback, or a visual alarm, e.g., light pattern, emitted directly by the tracking band 100 or by the device 20. Additionally, the electronic device 112 may be provided with motion charging components, such that each completed soleus pushup cycle may provide sufficient charging motion to enable and provide the energy for the operation of at least some of the foregoing operative features, e.g., light, speaker, haptic feedback, etc.

Yet additional features of the present invention comprise providing the tracking band 100 with a decorative component. It may be advantageous to provide for a receiver, such as a slot 115, that would allow a user 10 to swap or otherwise interchange a decorative component, e.g. a design, pattern, image, photograph, emblem, symbol, jewelry, group affiliation, social cause, ceremonial cause, organization, country, state, sports team, etc., to provide for multiple visual looks of the ankle mount 114, clip mount 117 and/or the electronic device 112 or cover thereof.

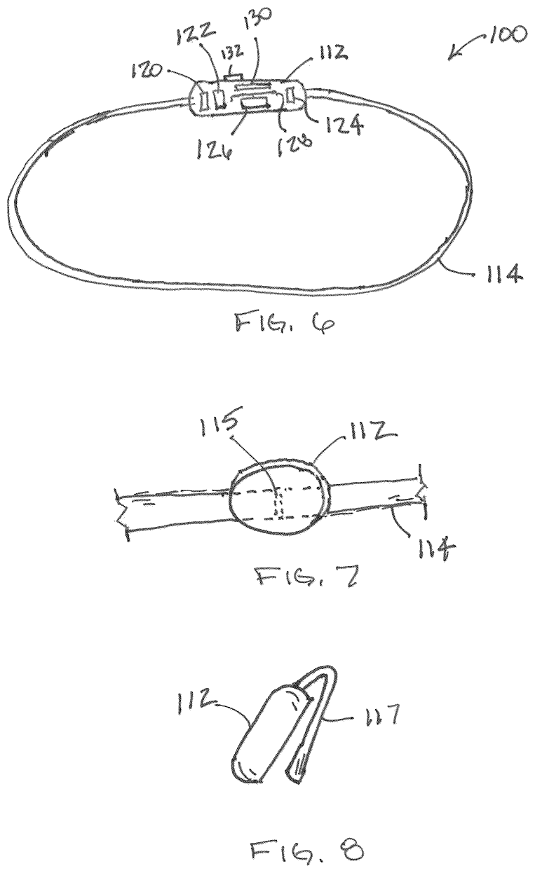

For example, the slot 115 may be disposed on, formed on, or connected or attached to the ankle mount 114 or clip mount 117 as shown in FIG. 7. Thus, a decorative component or cover of the electronic device 112, or a standalone decorative component, may be inserted into the slot 115 to change the visual appearance and style of the tracking band 100. Alternatively, a housing enclosing of the electronic device 112 may be configured and dimensioned to fit within the slot 115 so that it may inserted and removed. In turn, the slot 115 may comprise a geometry that allows for exposure of a decorative face of the electronic device 112 or housing thereof, while at the same time acting as a retaining component that secures it to the ankle mount 114 or clip mount 117.

Moreover, the electronic device 112, housing thereof, ankle mount 114 or clip mount 117 may comprise other attachment mechanisms that allow for the user 10 to removably attach the electronic device 112 or housing thereof to the ankle mount 114 or clip mount 117. These attachment mechanisms include, without limitation, brackets, clips, magnets, snap-on mechanisms, etc.

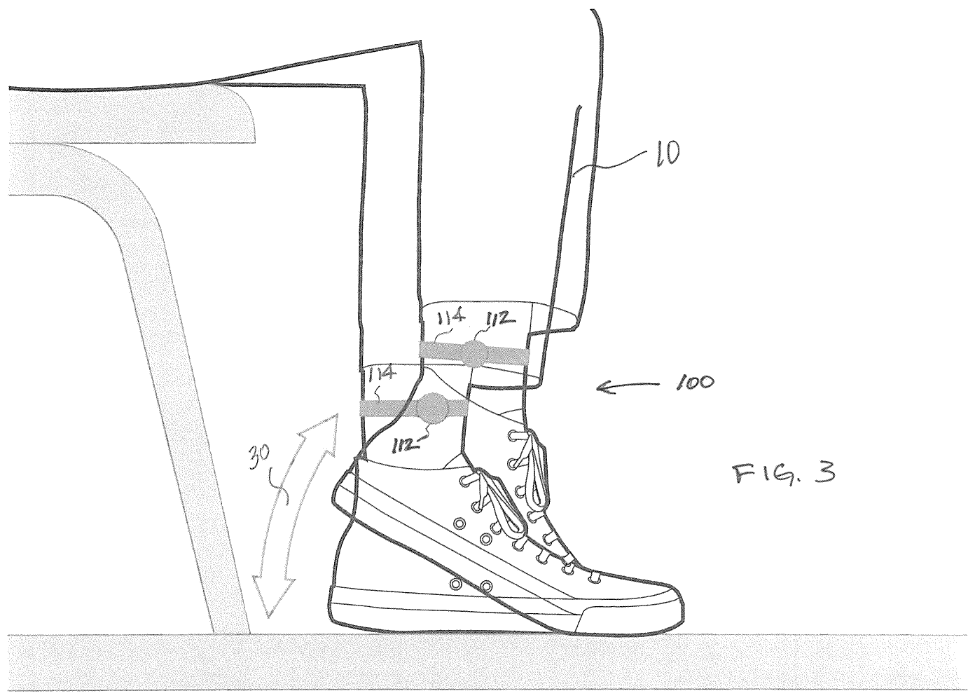

With reference now to FIG. 3 and briefly mentioned above, additional features of the present invention include an ancillary second tracking band. That is, the present invention generally contemplates that a first tracking band 100 be disposed on one corresponding ankle or ankle region of the user 10 in order to enable the operative features described herein. However, there are situations in which it may be desirable to ascertain and track the movement completed by both ankles of the user 10. In such situations, the present invention contemplates that an ancillary second tracking band 100 may be disposed on the other ankle of the user 10. Further, as may be appreciated from FIG. 3, each of the two tracking bands 100 may include a corresponding electronic device 112 each one having a sensor to independently track the movement of each ankle. Each of the two electronic devices 112 may be operatively configured or paired with the same device 20 to track the movement of both ankles.

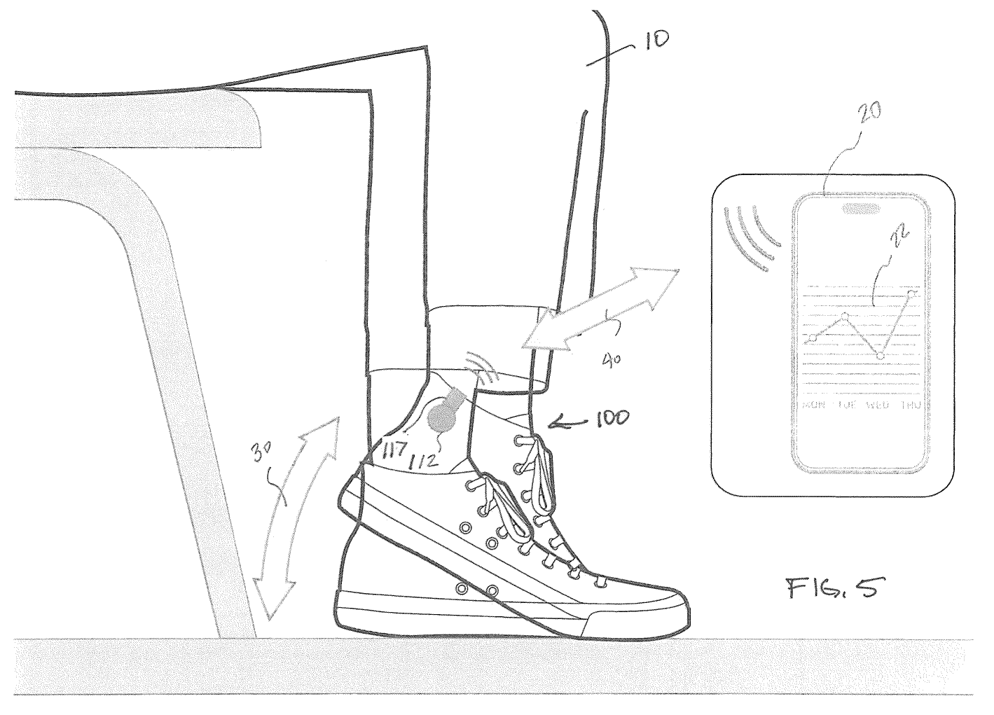

Referring now to FIG. 6, the tracking band detects and counts soleus pushup cycles using at least one sensor, primarily an accelerometer 120 and may include a gyroscope 122. The accelerometer 120 is configured to measure changes in movement and acceleration in three directions: forward/backward, up/down, and side-to-side. By tracking the movements and detecting the patterns associated with soleus pushup cycles, the tracking band 100 can estimate the number of soleus pushups the user 10 performed. The accelerometer 120 collects data continuously, monitoring changes in speed and direction. The tracking band 100 then uses this data to recognize patterns indicative of steps.

For example, the accelerometer 120 detects when the ankle moves and the tracking band 100 or device 20 calculates a soleus pushup when the movement matches stored calibration data of the pattern of a soleus pushup. In addition, when the pattern matches the pattern of walking or running then the movement is excluded from being counted as a soleus pushup. When a user walks or runs, the ankle moves in distinct, repetitive patterns. The accelerometer 120 captures this motion by detecting accelerations that correspond to each step. The accelerometer 120 collects data continuously, monitoring changes in speed and direction.

The gyroscope 122 is configured to determine the orientation and rotation of the tracking band 100 by detecting changes in the angle and position of the ankle of the user 10. The gyroscope 122 can also detect angular velocity to distinguish between different types of ankle movements.

The tracking band 100 refines the soleus pushup count by recognizing specific ankle motions of the user 10 that correspond to walking or running, improving accuracy. The data collected from the accelerometer 120 and gyroscope 122 may be processed by algorithms within the electronic device 112 or on the device 20. The algorithms use the motion data to filter out step movements of the user to estimate the number of soleus pushups more accurately. In particular, the gyroscope 122 coordinates with the accelerometer 120 to filter out walking and running movements of the user 10. The gyroscope 122 filters out movements other than the soleus pushup movement by recognizing leg swings that are characteristic of walking or running of the user 10, enhancing the accuracy by validating the motion detected by the accelerometer 120.

In addition, the tracking band 100 may include a GPS receiver 124 to further improve accuracy. The GPS receiver 124 tracks the amount of distance the user 10 has moved to determine if the user is walking or running rather than stationary, which can also improve soleus pushup counting.

The data from the accelerometer 120 and gyroscope 122 is processed by algorithms configured to recognize specific motion patterns associated with walking or running of the user 10 compared to a soleus pushup. The algorithms filter out irrelevant movements and focus on consistent patterns of the user 10. In addition, the tracking band 100 uses machine learning techniques to improve pattern recognition, learning from a vast dataset collected from different users, walking styles, soleus pushups, and environments. The processing is configured so that subtle or less-pronounced movements are still counted as performing a soleus pushup while reducing false positives.

Since the tracking band 100 is worn on the ankle or ankle region, it utilizes ankle specific motion data for soleus pushup counting. For example, when walking or running, the ankle tends to swing back and forth rhythmically. The tracking band 100 detects this movement pattern, which helps identify the timing of each step and filter out that movement from counting as a soleus pushup. Also, by determining the cadence and the rhythm of the detected ankle movements of the user 10, the tracking band 100 can distinguish between a soleus pushup, walking, and running of the user 10.

The tracking band 100 is configured to use certain motion thresholds to determine whether a movement qualifies as a soleus pushup. For example, if the detected acceleration is too high or the movement is too long for the user 10, it might be ignored. The algorithms also consider the acceleration profile of the user 10, which includes factors like length of ankle movement and movement frequency, to classify the soleus pushup accurately.

The tracking band 100 is configured to be calibrated to adapt to the soleus pushup patterns of a particular user over time. When the user 10 wears the tracking band 100 consistently, the tracking band learns the soleus pushup movement compared to the walking style, stride length, and typical step frequency of the user 10.

If the user 100 is bicycling, walking or running with the GPS receiver 124 enabled on the tracking band 100, the tracking band 100 is configured to compare the detected movement with the actual distance traveled. This data is used to determine whether a soleus pushup was performed if GPS indicates the user was stationary, or if a distance traveled was detected then the user was engaged in a different activity than a soleus pushup. Calibration allows the tracking band 100 to adjust to different movements of the user 10 making soleus pushup detection more accurate.

The electronic device 112 may also include a wireless communication transceiver and circuitry 126 coupled to one or more antennas 128 for communicating with the mobile device 20 discussed above, and depicted in FIG. 6. The antennae 128 may be a short-range wireless antenna, a near-field antenna, or a GPS antenna, for example. The accelerometer 120, gyroscope, GPS receiver 124 and any other sensors are powered by a rechargeable battery 130. A haptic device 132 may be included with the electronic device 112. Accordingly, the haptic device 132 may be configured to vibrate to signal to the user 10 that the electronic device 112 is communicating with the mobile device 20. The haptic device 132 may also be configured to vibrate with each soleus pushup to indicate to the user 10 that the movement was counted. Motion sensors of other types might be employed. Ball motion sensors, piezo sensors, Hall effect sensors, even liquid mercury switches, etc. could be potentially utilized.

Referring now to FIG. 9, a method 200 implemented in a tracking band wearable on the ankle of a user is depicted. The method 200 begins at 202 and includes, at 202, detecting movement by an electronic device of the tracking band. The method also includes transmitting sensor data of the movement to a mobile device, at 204. In addition, the method 200 includes analyzing the sensor data to determine if a soleus pushup was performed, at 208. If the sensor data suggests that a soleus pushup was performed, then the soleus pushup count is increased, at 210, and the method returns to detecting motion, at 204. If the sensor data suggests that a soleus pushup was not performed, then the movement is disregarded, at 212, and the method returns to detecting motion, at 204. When no motion is detected, at 204, the method ends, at 214.

The previous description of the disclosed embodiments is provided to enable any person skilled in the art to make or use the disclosed embodiments. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope possible consistent with the principles and novel features as defined herein.

1. An ankle movement tracking band comprising:
an electronic device configured to be worn on an ankle region of a user, the electronic device comprising at least one motion sensor configured to capture sensor data of movement of the ankle region of the user; and
a processor coupled to a memory for storing calibration data, the processor configured to receive and analyze the sensor data to determine whether the sensor data matches the calibration data indicating the user performed a soleus pushup.

2. The ankle movement tracking band of claim 1, wherein the at least one motion sensor comprises one of an accelerometer, a gyroscope, and a Global Positioning System (GPS) receiver.

3. The ankle movement tracking band of claim 1, wherein the electronic device further comprises a haptic device.

4. The ankle movement tracking band of claim 1, wherein the electronic device further comprising a wireless communication transceiver configured to connect to a device and transmit the sensor data to the device.

5. The ankle movement tracking band of claim 4, wherein the device is configured to connect to a network to transmit the sensor data to a remote device for processing.

6. The ankle movement tracking band of claim 5, wherein the electronic device further comprises an antenna coupled to the wireless communication transceiver.

7. The ankle movement tracking band of claim 1, further comprising an ankle mount, the electronic device secured to the ankle mount.

8. The ankle movement tracking band of 7, wherein the ankle mount includes a receiver for removably attaching a decorative component to cover the electronic device.

9. The ankle movement tracking band of claim 1, wherein the device having a soleus pushup counter module configured to count a number of soleus pushups performed by the user when the sensor data matches the calibration data.

10. The ankle movement tracking band of claim 3, wherein the haptic device is configured to vibrate in response to receiving an alert from the processor.

11. The ankle movement tracking band of claim 1, further comprising an ancillary second electronic device configured to be worn on an other ankle region of the user, the ancillary second electronic device having at least one respective motion sensor configured to capture respective sensor data of movement of the other ankle of the user.

12. The ankle movement tracking band of claim 7, wherein the ankle mount comprises a strap configured to encircle the ankle of the user or a clip configured to secure to clothing proximate to the ankle of the user.

13. The ankle movement tracking band of claim 1, wherein the calibration data correlates to vertical, oscillatory movements of performing the soleus pushup.

14. An ankle movement tracking system, the system comprising:
a tracking band comprising,
an electronic device configured to be worn on an ankle region of a user, the electronic device having at least one motion sensor configured to capture sensor data of movement of the ankle region of the user; and
a wireless communication transceiver configured to wirelessly transmit the sensor data and receive commands and data; and
a device having a processor coupled to a memory for storing calibration data, the device configured to receive the sensor data from the tracking band via the wireless communication transceiver, and to analyze the sensor data to determine whether the sensor data matches the calibration data indicating the user performed a soleus pushup.

15. The ankle movement tracking system of claim 14, wherein the at least one motion sensor comprises one of an accelerometer, a gyroscope, and a Global Positioning System (GPS) receiver.

16. The ankle movement tracking system of claim 15, wherein the device further comprising a soleus pushup counter module configured to increase a count of a number of soleus pushups performed by the user correlating to when the sensor data matches the calibration data.

17. The ankle movement tracking system of claim 16, further comprising an ancillary second electronic device configured to be worn on an other ankle region of the user, the ancillary second electronic device having at least one respective motion sensor configured to capture respective sensor data of movement of the other ankle region of the user.

18. A method of tracking ankle movement of a user, the method comprising:
placing a tracking band on an ankle region of the user;
detecting movement by an electronic device of the tracking band;
transmitting sensor data of the movement to a device;
analyzing the sensor data to determine if a soleus pushup was performed;
increasing a count of a soleus pushup count when the sensor data matches with calibration data that the soleus pushup was performed; and
disregarding the sensor data when the sensor data does not match with the calibration data the soleus pushup was not performed.

19. The method of claim 18, further comprising placing a second tracking band on an other ankle region of the user.

20. The method of claim 18, further comprising displaying the soleus pushup count on the device.
